(19) United States
(12) Patent Application Publication
(10) Pub. No.: US 2010/0150955 A1
(43) Pub. Date: Jun. 17, 2010

(54) Method of Producing Extract Derived From Lyophyllum Ulmarium

(76) Inventors: Hiroshi Endo, Shiga (JP); Hitoshi Sakakibara, Shiga (JP); Yasushi Hattori, Shiga (JP); Kumi Sasaki, Shiga (JP); Ikunoshin Kato, Shiga (JP)

Correspondence Address: BIRCH STEWART KOLASCH & BIRCH, PO BOX 747, FALLS CHURCH, VA, 22040-0747, US

(21) Appl. No.: 11/990,125

(22) PCT Filed: Aug. 2, 2006
(86) PCT No.: PCT/JP2006/315311

§ 371 Date: Feb. 7, 2008

(30) Foreign Application Priority Data
Aug. 9, 2005 (JP) 2005-230641

(51) Int. Cl. A61K 36/06; A23L 1/28

(52) U.S. Cl. 424/195.15; 426/429

(57) ABSTRACT

A method of producing an extract derived from Lyophyllum ulmarium from fruiting body or mycelium of Lyophyllum ulmarium, wherein the method of producing an extract derived from Lyophyllum ulmarium uses an aqueous alkaline solvent as an extraction solvent; an extract derived from Lyophyllum ulmarium, obtainable by the method as defined above; and a food and a medicament each containing the extract.

### TECHNICAL FIELD

The present invention relates to a method of producing an extract derived from Lyophyllum ulmarium, an extract derived from Lyophyllum ulmarium obtainable by the method, a food containing the extract, and a medicament containing the extract. Further, the present invention relates to a method of producing a terpene compound derived from Lyophyllum ulmarium.

### BACKGROUND ART

In recent years, search of physiologically active substances from Basidiomycetes has been extensively carried out, and various reports have been made thereon. As a physiologically active component derived from Lyophyllum ulmarium, a substance named SBS has been known to possess suppressive action on platelet aggregation and action of anti-tumor promotion (for example, Patent Publication 1). This SBS is one kind of a terpene compound (terpenoid), which is a bitterness component, and it is known that Lyophyllum ulmarium contains various terpene compounds besides the above (for example, Non-Patent Publication 1).

Terpene compounds are organic compounds existing in a wide variety of plants, of which number of carbon atoms is a multiple of 5, and the terpene compounds are derived from precursor substances composed of n number of isoprene or isopentane. Those satisfying n=2 are named as monoterpenes; those satisfying n=3 are named sesquiterpenes, those satisfying n=4 are named diterpenes, those satisfying n=5 are named sesterterpenes, those satisfying n=6 are named triterpenes, those satisfying n=8 are named tetraterpenes, and those satisfying n above that value are named polyterpenes. The terpene compounds have been known to have an action of inducing apoptosis. For example, there are numerous reports, including a report on an action of inducing apoptosis by a triterpene, and the like.

Terpene compounds derived from Lyophyllum ulmarium have been known to be hardly soluble in water (for example, Patent Publication 1). As a method of extracting the terpene compound, a method including the step of extracting a terpene compound with an organic solvent such as ethyl acetate from a fruiting body of Lyophyllum ulmarium (for example, Patent Publication 1), or a method including the step of extracting a terpene compound with a 70% ethanol from a heat-treated fruiting body of Lyophyllum ulmarium (for example, Non-Patent Publication 1) has been known.

Patent Publication 1: Japanese Patent Laid-Open No. Hei 4-104795
Non-Patent Publication 2: Sawabe A. and three others, Journal of Mass Spectrometry, 1996, 31, 921-925

### DISCLOSURE OF THE INVENTION

### Problems to be Solved by the Invention

In a case where a terpene compound is extracted from Lyophyllum ulmarium in accordance with the conventional methods as mentioned above, a large amount of an organic solvent, for example, ethanol or ethyl acetate must be used. Meanwhile, ethanol or ethyl acetate is handled as a hazardous substance, so that upon production on an industrial scale, sufficient care is required in its handling as well as maintenance, control, use and the like, from the viewpoint of safety. In addition, equipments for maintaining and controlling these hazardous substances would be necessitated. These factors are synergistically acted to cause rise in the cost in the production of an extract derived from Lyophyllum ulmarium richly containing the terpene compound, which is a serious problem upon the production on an industrial scale.

In addition, when the extract derived from Lyophyllum ulmarium obtained by the conventional method as mentioned above is used for foods, there is a risk of having a disadvantage that the residual organic solvent in the extract spoils the flavor of the food, and causing harmful influences to human bodies in a case where the organic solvent is a toxic substance. Even if the step of removing the residual organic solvent is added in order to avoid these disadvantages, it is difficult to completely remove the residual organic solvent. Also, by adding the step of removing the organic solvent, the production cost would be further raised.

Accordingly, an object of the present invention is to provide an extract derived from Lyophyllum ulmarium richly containing a terpene compound by efficiently extracting the terpene compound from Lyophyllum ulmarium without using an organic solvent at a low cost.

### Means to Solve the Problems

The present inventors have found that an extract derived from Lyophyllum ulmarium richly containing a terpene compound can be produced without using an organic solvent, by processing Lyophyllum ulmarium with an alkaline extraction solvent. The present invention has been perfected thereby.

Specifically, summarizing the present invention, a first invention of the present invention relates to a method of producing an extract derived from Lyophyllum ulmarium from fruiting body or mycelium of Lyophyllum ulmarium, wherein the method of producing an extract derived from Lyophyllum ulmarium uses an aqueous alkaline solvent as an extraction solvent. In the first invention, the method may further include the step of neutralizing the resulting extract with an acid. In addition, in the first invention, the method may further include the step of adding an emulsifying agent.

The aqueous alkaline solvent usable in the first invention is exemplified by an aqueous sodium carbonate solution, an aqueous sodium hydroxide solution, or an anionic exchange resin suspension.

In the first invention, the extraction with an extraction solvent is exemplified by an extraction at a temperature of from 40° to 120° C.

A second invention of the present invention relates to an extract derived from Lyophyllum ulmarium, obtainable by the method of the first invention. The extract from Lyophyllum ulmarium of the second invention is exemplified by an extract containing a terpene compound in an amount of 0.4% by weight or more on a dry solid basis.

A third invention of the present invention relates to a food containing the extract derived from Lyophyllum ulmarium of the second invention.

A fourth invention of the present invention relates to a medicament containing the extract derived from Lyophyllum ulmarium of the second invention.

A fifth invention of the present invention relates to a method of producing a terpene compound, including the steps of:

(a) processing a fruiting body or mycelium of Lyophyllum ulmarium with an alkaline extraction solvent, to give an extract derived from Lyophyllum ulmarium; and
(b) purifying the terpene compound from the extract obtained in the step (a).

The aqueous alkaline solvent usable in the fifth invention is exemplified by an aqueous sodium carbonate solution, an aqueous sodium hydroxide solution, or an anionic exchange resin suspension.

In the fifth invention, the processing with an aqueous alkaline solvent is exemplified by carrying out a processing at a temperature of from 40° to 120° C.

### EFFECTS OF THE INVENTION

According to the present invention, a method of producing an extract derived from Lyophyllum ulmarium richly containing a terpene compound is provided. Since the method does not necessitate an organic solvent during the production, an extract suitable for foods can be produced. Further, as compared to the conventional production methods, the production costs can be reduced. In addition, according to the method, an extract derived from Lyophyllum ulmarium suitable for foods, the extract richly containing the terpene compound is provided. Also, according to the present invention, a food and a medicament, each containing the extract derived from Lyophyllum ulmarium, are provided. Furthermore, an inexpensive method of producing a terpene compound is provided.

### BEST MODE FOR CARRYING OUT THE INVENTION

Lyophyllum ulmarium grows in nature in caespitose or scattered state on leafless trees of various broad-leaved trees in the autumn season. As compared to other mushrooms, Lyophyllum ulmarium is excellent in its shape and has crisp fleshy substance, so that the Lyophyllum ulmarium has been harvested to be eaten as delicious mushroom. Also, in the recent years, a method of artificially cultivating on a bed including the step of cultivating in a bottle or box using a culture substrate containing sawdust formulated with rice bran and other nutrients has been established, and the mushrooms can presently be harvested stably through the year regardless of the season. In other words, Lyophyllum ulmarium, which is used as a raw material for the extract of the present invention, can be made available inexpensively, so that Lyophyllum ulmarium is suitable as a raw material for medicaments or health-care foods.

The Lyophyllum ulmarium, which is used as a raw material, may be a natural product or an artificially cultivated product, and is preferably exemplified by Lyophyllum ulmarium M-8171 (FERM BP-1415, date of deposit: Aug. 23, 1986), or Lyophyllum ulmarium K-0259 (FERM P-12981, date of deposit: Jun. 2, 1992) (these strains are deposited with the International Patent Organism Depositary, National Institute of Advanced Industrial Science and Technology, of which the address is AIST Tsukuba Central 6, 1-1, Higashi 1-chome, Tsukuba-shi, Ibaraki-ken 305-8566, Japan). In addition, these strains are distributed in the market under the trade name of “Yamabiko Honshimeji (registered trademark)” or “Super-Yamabiko Shimeji (registered trademark).” In a case where an artificially cultivated product is used as Lyophyllum ulmarium, a fruiting body of Lyophyllum ulmarium cultivated in a medium containing a substance that improves the content of the terpene compound in the extract derived from Lyophyllum ulmarium produced, for example, bean coat, is preferably used. The fruiting body can be used as a raw material in the form as it is in the form of cluster, or after being powdered, and the fruiting body can be used as a raw material in the form of fresh product, or a dried product of the fruiting body by means of heated air drying, drying in the sun, lyophilization or the like. Also, as the raw material, the mycelium of Lyophyllum ulmarium or a lyophilized product thereof can be used as the raw material. In addition, a product obtained by washing the above-mentioned fruiting body or mycelium with hot water can be used as the raw material.

By using an aqueous alkaline solvent as an extraction solvent, the present inventors have succeeded in the production of an extract derived from Lyophyllum ulmarium richly containing a terpene compound that is hardly soluble in water, without using an organic solvent. Here, in the present invention, the terpene compound is exemplified by a polyterpene derived from Lyophyllum ulmarium as described in the above-mentioned Non-Patent Publication 1.

The extraction solvent used in the method of producing an extract derived from Lyophyllum ulmarium of the present invention is not particularly limited, as long as the extraction solvent is an aqueous alkaline solvent without containing an organic solvent. For example, an aqueous alkaline solution containing an inorganic base (for example, an alkali metal hydroxide such as sodium hydroxide or potassium hydroxide; an alkaline earth metal hydroxide such as magnesium hydroxide or barium hydroxide; an alkali metal carbonate such as sodium carbonate or potassium carbonate; an alkaline earth metal carbonate such as magnesium carbonate or barium carbonate; an alkali metal hydrogencarbonate such as sodium hydrogencarbonate or potassium hydrogencarbonate; ammonium carbonate; ammonia; or the like); an aqueous alkaline solution containing an organic base (for example, an organic acid salt of an alkali metal, such as sodium acetate or potassium propionate; an organic acid salt of an alkaline earth metal, such as magnesium formate or magnesium acetate; an amine such as trimethylamine, ethylamine, diethylamine, triethylamine, propylamine, butylamine, or piperidine; a nitrogen-containing heterocyclic compound such as pyridine; or the like); and an alkaline suspension containing an anionic exchange resin can be used. Among them, an aqueous sodium carbonate solution, an aqueous sodium hydroxide solution, or an anionic exchange resin suspension can be preferably used.

Further, since precipitates derived from the extract are less likely to be formed when the extract is neutralized and the extract after the neutralization is less likely to be foamed, an anionic exchange resin suspension can be more preferably used.

The content of the inorganic base, the organic base, or the anionic exchange resin in the extraction solvent is not particularly limited, and can be adjusted so that the extraction solvent has a pH that falls within the range given below.

In addition, the pH of the extraction solvent is not particularly limited so long as the extraction solvent shows alkalinity, and the pH is preferably from 8 to 12, and more preferably from 9 to 12.

The processing temperature with the extraction solvent is not particularly limited, so long as the temperature is a temperature at which a terpene compound can be extracted in a desired amount. For example, the processing can be carried out at a temperature of from 4° to 120° C., and it is preferable that the processing is carried out at a high temperature, from the aspect that an even more amount of the terpene compound can be extracted in a shorter time period. The processing temperature as mentioned above is preferably from 40° to 120° C., more preferably from 80° to 110° C., even more preferably from 85° to 105° C., and most preferably from 90° to 100° C.

The processing time with the extraction solvent differs depending upon the processing temperature. For example, it is preferable that the processing time is from 16 to 24 hours, in a case where the processing temperature is from 4° to 60° C., and that the processing time is from 0.5 to 5 hours, in a case where the processing temperature is from 90° to 105° C.

The method of producing an extract derived from Lyophyllum ulmarium further including the step of neutralizing the extract obtained by the above-mentioned method with an acid is also encompassed in the method of producing an extract derived from Lyophyllum ulmarium of the present invention. As the acid used in the neutralization, in a case where the extract is used for foods, an acid usable as a food additive is preferable. The acid usable as a food additive includes, for example, adipic acid, citric acid, gluconic acid, succinic acid, acetic acid, and tartaric acid. Among them, citric acid is most preferable.

In addition, it is preferable that the above-mentioned method further includes the step of adding a proper emulsifying agent. By the stabilization with an emulsifying agent, the precipitation of the terpene compound that is hardly soluble in water can be controlled, and the adsorption of the terpene compound to machinery equipments and side materials in the production steps can also be controlled. The timing of the addition of the emulsifying agent may be any of the timing of before the extraction, after the extraction, or in the course of the extraction with an aqueous alkaline solvent. For example, as described in Example 4 set forth below, it is preferable that the emulsifying agent is added after the extraction. In addition, it is preferable that the above-mentioned neutralizing step is carried out after the addition of the emulsifying agent. The emulsifying agent usable in the present invention is not particularly limited. For example, a polyglycerol fatty acid ester, a sorbitan fatty acid ester, a propylene glycol fatty acid ester, calcium stearoyl lactate, a soybean-derived lecithin, an egg yolk-derived lecithin, or the like can be used.

The amount of the emulsifying agent is not particularly limited, and it is preferable that the amount is, for example, from 0.1 to 3 parts by weight, based on 100 parts by weight of the extract.

The extract derived from Lyophyllum ulmarium of the present invention can be obtained by further removing extraction residue from the above extract obtained by the above-mentioned method. The step of removing extraction residue is exemplified by centrifugation, filtration, or ultrafiltration, and a filtration method that is suitable for a large-scale production can be more preferably used. Upon filtration, a filtration aid can be used, and the filtration aid is exemplified by Celite #545.

In the present invention, the extract refers to a substance that is obtained by the step of carrying out an extraction procedure with an extraction solvent. Also, a substance obtained by further subjecting the above-mentioned substance to such a process as filtration, centrifugation, concentration, ultrafiltration, molecular sieving or neutralization is also encompassed in the extract of the present invention. In addition, a fraction obtained by fractionating the above-mentioned substance by a known method, or a fraction obtained by repeating the fractionation procedures a plural times is also encompassed in the extract of the present invention. The above-mentioned fractionation means includes extraction, separation by precipitation, column chromatography, thin-layer chromatography, and the like.

In the present invention, the shape of the extract of the present invention is not particularly limited, and the extract may take any form of powder, solid or liquid. When the extract is used in the form of powder, the extract of the present invention can obtained by concentrating an extract obtained by extracting the raw material with an aqueous alkaline solvent, further adding an excipient such as dextrin, a sucrose fatty acid ester, or lactose thereto, drying the mixture, and powdering the residue, without particularly being limited thereto. In addition, a granular solid obtained by granulating the extract by a known process can be used as the extract of the present invention. The granulation process is not particularly limited, and is exemplified by tumbling granulation, agitation granulation, fluidizing bed granulation, airflow granulation, extruding granulation, compression molding granulation, disintegration granulation, spray granulation, spray-drying granulation or the like. In addition, the extract in the form of a liquid is exemplified by a liquid as it is, obtained by the method of producing the above-mentioned extract, a concentrate thereof, or a dilution thereof, as well as one liquefied by dissolving the above-mentioned powdery extract in a liquid, for example, water, an alcohol or the like to give a liquid.

The feature of the extract derived from Lyophyllum ulmarium of the present invention is in that the extract, contains in a large amount a terpene compound such as SBS, which is a physiologically active component derived from Lyophyllum ulmarium. For example, the extract of the present invention is an extract containing a terpene compound in an amount of 0.4% by weight or more on a dry solid basis, and more specifically, an extract containing a terpene compound in an amount of from 0.4 to 1.0% by weight on a dry solid basis. The phrase “% by weight on a dry solid basis” refers to a value expressing the weight of the intended component in percentage based on the entire dry product obtained when the liquid is dried with an evaporator or the like.

The food of the present invention contains the above-mentioned extract derived from Lyophyllum ulmarium. Since the food of the present invention richly contains a terpene compound derived from Lyophyllum ulmarium, such as SBS, the food can be expected to possess suppressive action for platelet aggregation, action of anti-tumor promotion, and carcinostatic action. The food can also serve as a health food indicated to be used for the exhibition of the desired effect caused by, for example, suppressive action for platelet aggregation, action of anti-tumor promotion, or carcinostatic action (food for specified health uses).

The process for producing the food of the present invention is not particularly limited. For example, combination, cooking, processing, and the like can be carried out in accordance with those general foods, and the food of the present invention can be produced by the general methods, as long as the resulting food contains the above-mentioned extract according to the present invention. In addition, the extract derived from Lyophyllum ulmarium of the present invention per se may be used as a food.

The term “comprising” in the food of the present invention encompasses the meanings of containing(ed), adding(ed) and/or diluting(ed). As used herein, the term “containing(ed)” refers to an embodiment of containing the extract used in the present invention in the food; the term “adding(ed)” refers to an embodiment of adding the extract used in the present invention to a raw material for the food; and the term “diluting(ed)” refers to an embodiment of adding a raw material for the food to the extract used in the present invention.

The food of the present invention is not particularly limited. The food includes, for example, processed grain products (for example, processed wheat products, processed starch products, processed premix products, noodles, macaronis, bread, bean jam, buckwheat noodles, wheat-gluten bread, rice noodles, fen-tiao, and packed rice cake); processed fat and oil products (for example, plastic fat and oil, tempura oil, salad oil, mayonnaise, and dressing); processed soybean products (for example, tofu products, soybean paste (miso), and fermented soybeans); processed meat products (for example, ham, bacon, pressed ham, and sausage); marine products (for example, frozen ground fish, steamed fish paste, tubular roll of steamed fish paste, cake of ground fish, deep-fried patty of fish paste, fish ball, fascia and tendon, fish meat ham, sausage, dried bonito, products of processed fish egg, canned marine products, and preserved food boiled down in soy sauce (tsukudani)); milk products (for example, raw material milk, cream, yogurt, butter, cheese, condensed milk, powder milk, and ice cream); processed vegetable and fruit products (for example, paste, jam, pickled vegetables, fruit beverages, vegetable beverages, and mixed beverages); confectionaries (for example, chocolates, biscuits, sweet bun, cake, rice cake snacks and rice snacks); alcohol beverages (for example, sake, Chinese liquor, wine, whiskey, Japanese distilled liquor (shochu), vodka, brandy, gin, rum, beer, refreshing alcoholic beverages, fruit liquor, and liqueur); luxury drinks (for example, green tea, tea, oolong tea, coffee, health-care beverages, refreshing beverages and lactic acid beverages); seasonings (for example, soy sauce, sauce, vinegar, and sweet rice wine); canned, bottled or pouched foods; semi-dry or concentrated foods (for example, liver pastes and other spreads, soups for buckwheat noodles or wheat noodles, and concentrated soups); dry foods (for example, instant noodles, instant curry roux, instant coffee, powder juice, powder soup, instant soybean paste (miso) soup, precooked foods, precooked beverages, and precooked soup); frozen foods; and the like.

The food of the present invention is not particularly limited in its shape, as long as the above-mentioned extract is contained. The shape includes those shapes that can be taken orally such as tablets, granules and capsules. In addition, glycerol may be added to the extract mentioned above, whereby a health food in which the above-mentioned extract is concentrated can be provided.

The content of the above-mentioned extract in the food of the present invention is not particularly limited, and the content can be properly set from the viewpoints of sensory aspect and exhibition of activity. For example, the content is from 0.1 to 100% by weight, preferably from 0.5 to 95% by weight, and even more preferably from 1 to 90% by weight, of the food, on a dry basis of the above-mentioned extract.

Since the food of the present invention is capable of controlling bitterness of the extract derived from Lyophyllum ulmarium and mildening the flavor, it is preferable in the method of producing an extract mentioned above that an extract derived from Lyophyllum ulmarium obtained by adding an emulsifying agent is used.

Here, as the food of the present invention, a beverage as described in Example 4 is especially preferable from the aspect that the absorption of the terpene compound into the body is excellent.

The medicament of the present invention contains an extract derived from Lyophyllum ulmarium as mentioned above. The medicament of the present invention includes ones formed into a preparation by combining the above-mentioned extract according to the present invention with a known pharmaceutical carrier.

The medicament of the present invention is usually manufactured by combining the above-mentioned extract with a pharmaceutically acceptable liquid or solid carrier. A solvent, a dispersant, an emulsifying agent, a buffer, a stabilizer, an excipient, a binder, a disintegrant, a lubricant, or the like can be optionally added thereto, to form a solid agent such as a tablet, a granule, a powder, an epipastic, and a capsule, or a liquid agent such as a common liquid agent, a suspension agent or an emulsion agent. In addition, there can be also formed into a dry product which can be liquefied by adding an appropriate carrier before use, or also into an external preparation.

The pharmaceutical carrier can be selected depending upon the administration form and preparation form of the medicament of the present invention. In the case of an orally administered preparation comprising a solid composition, the preparation can be produced in the form of a tablet, a pill, a capsule, a powder, a fine powder, a granule or the like; for example, starch, lactose, saccharose, mannitol, carboxymethyl cellulose, cornstarch, an inorganic salt or the like can be utilized as a carrier. In addition, upon preparation of the orally administered preparation, a binder, a disintegrant, a surfactant, a lubricant, a fluidity accelerator, a corrective, a colorant, a flavor, and the like can be further combined therewith. In the case of forming into a tablet or a pill, for example, the tablet or pill may be covered with a sugar-coating made of sucrose, gelatin or hydroxypropyl cellulose, or with a film made of a substance soluble in the stomach or intestine as desired. In the case of an orally administered preparation comprising a liquid composition, the preparation can be prepared in the form of a pharmaceutically acceptable emulsion, solution, suspension, syrup, or the like. In this case, for example, purified water, ethanol or the like is utilized as a carrier. Furthermore, an auxiliary agent such as a wetting agent or a suspending agent, a sweetener, a flavor, an antiseptic, or the like may be added as desired. Here, since the extract of the present invention exhibits a sufficient effect even by oral administration, its preferred form is a medicament for oral administration, from the viewpoint of convenience of the administration.

On the other hand, in the case of a parenterally administered preparation, the preparation can be prepared by dissolving or suspending the above-mentioned extract of the present invention in a diluent such as distilled water for injection, physiological saline, an aqueous solution of glucose, vegetable oil for injection, sesame oil, peanut oil, soybean oil, corn oil, propylene glycol or polyethylene glycol, in accordance with a conventional method, and adding a microbicide, a stabilizer, a tonicity agent, a soothing agent, or the like if needed. It is also possible to produce a solid composition and dissolve the composition in sterile water or a sterile solvent for injection before use.

The external preparation includes solid, semi-solid or liquid preparations for percutaneous administration or transmucosal (intraoral or intranasal) administration. The external preparation also includes suppositories and the like. For example, the external preparation can be prepared as liquid preparations including emulsions, suspensions such as lotions, external tinctures, and liquid agents for transmucosal administration; ointments such as oily ointments and hydrophilic ointments; patches for percutaneous administration or transmucosal administration such as films, tapes and poultices; and the like.

Each of the above-mentioned various preparations can be appropriately produced in accordance with conventional methods by utilizing known pharmaceutical carriers and the like. Also, the content of the extract in the preparation is not particularly limited, as long as the content is preferably in an amount so that the extract can be administered within the dose range described below in consideration of administration form, administration method and the like of the preparation. The content of the above-mentioned extract in the medicament of the present invention is usually from 0.1 to 100% by weight or so on a dry basis.

The dose of the medicament of the present invention is changeable and properly set depending upon its preparation form, administration method, purpose of use, and age, body weight, symptom or the like of a patient to which the medicament is administered. Generally, the dose of the medicament, in terms of the dose of the above-mentioned extract contained in the preparation when expressed on a dry basis, is from 1 μg to 100 mg/kg body weight, preferably from 5 μg to 50 mg/kg body weight, and even more preferably from 10 μg to 10 mg/kg body weight, per day for adult. As a matter of course, the dose varies depending upon various conditions, so that an amount smaller than the dose mentioned above may be sufficient, or an amount exceeding the dose range may be required. Administration may be carried out once or in several divided portions in a day within the desired dose range. The administration period may be arbitrarily determined. Also, the medicament of the present invention can be orally administered as it is, or the medicament can be added to any foodstuffs to be taken on a daily basis.

The method of producing a terpene compound of the present invention is a method of producing a terpene compound including the steps of (a) processing a fruiting body or mycelium of Lyophyllum ulmarium with an aqueous alkaline solvent, to give an extract derived from Lyophyllum ulmarium; and (b) purifying the terpene compound from the extract obtained in the step (a). In the method, the extract obtained by the step (a) is exemplified by the extract derived from Lyophyllum ulmarium of the present invention as mentioned above. In addition, in the method of producing the terpene compound, as in the method of producing an extract derived from Lyophyllum ulmarium of the present invention, as an aqueous alkaline solvent, an aqueous sodium carbonate solution, an aqueous sodium hydroxide solution, or an anionic exchange resin suspension can be preferably used, and the processing temperature with the extraction solvent is not particularly limited as long as the processing temperature is a temperature at which the terpene compound can be extracted in a desired amount; for example, the processing can be carried out at 4° to 120° C. Since the method of producing a terpene compound of the present invention derived from Lyophyllum ulmarium of the present invention does not necessitate an organic solvent in the step of extracting an extract richly containing the terpene compound from a fruiting body or mycelium of Lyophyllum ulmarium, the terpene compound can be produced inexpensively.

The terpene compound derived from Lyophyllum ulmarium which can be produced according to the method of producing a terpene compound of the present invention includes a substance SBS described in Japanese Patent Laid-Open No. Hei 4-104795. This substance is identical to a substance hypsiziprenol A₉, which is described in a publication by Sawabe et al. (Sawabe A. and three others, Journal of Mass Spectrometry, 1996, 31, 921-925). In addition, the terpene compounds which can be produced according to the method of producing a terpene compound of the present invention other than SBS are exemplified by terpene compounds derived from Lyophyllum ulmarium, such as hypsiziprenol A₈, A₁₀, A₁₁, A₁₂, A₁₃, A₁₄, B₈, B₉, B₁₀, B₁₂, and C₉, which are described in the above-mentioned publication by Sawabe et al.

As a method of purifying a terpene compound from an extract obtainable by processing Lyophyllum ulmarium with an aqueous alkaline solvent, a known method can be employed, and the method includes extraction, separation by precipitation, column chromatography, thin-layer chromatography, and combinations of these methods. The column chromatography is exemplified by high-performance liquid chromatography, and in the present invention, reversed phase high-performance liquid chromatography can be preferably employed.

### EXAMPLES

The present invention will be specifically described hereinbelow by the Examples, without intending to limit the scope of the present invention thereto.

### Preparation Example 1

### Preparation of Standard Substance

In accordance with the method described in Example 1 of Japanese

Patent Laid-Open No. Hei 4-104795, the procedures of extraction of an extract derived from Lyophyllum ulmarium, purification of the extract with a silica gel column, and fractionation by reversed phase high-performance liquid chromatography were carried out. Among the fractions obtained, a fraction G and a fraction containing SBS was analyzed by using HPLC/MS method. It was confirmed that the purified products from these two fractions are terpene compounds each having a different structure. Here, a fraction G was a substance identical to hypsiziprenol A_{g} described in the publication by Sawabe et al. (Sawabe A. and three others, Journal of Mass Spectrometry, 1996, 31, 921-925). In addition, the SBS was a substance identical to hypsiziprenol A₉ described in the above-mentioned publication by Sawabe et al. The purified products of the fraction G and the SBS thus obtained were, named a standard substance hypsiziprenol A₈ and SBS, each of which is a terpene compound derived from Lyophyllum ulmarium.

### Example 1

### Extraction 1 with Alkaline Solvent

Fruiting bodies of Lyophyllum ulmarium (Lyophyllum ulmarium M-8171) were sufficiently dried in a Constant Temperature Oven (manufactured by Yamato), and the dried product was then powdered with a Cooking Mill (manufactured by NATIONAL PANASONIC), to give a dry powder of Lyophyllum ulmarium. Next, 0.1 L of a 1% aqueous Na₂CO₃ solution (pH 11.93) was added to 5 g of the dry powder of Lyophyllum ulmarium, and the mixture was heat-treated at 95° C. for 3 hours. After the heat treatment, the heat-treated mixture was subjected to a centrifugation procedure at 10,000 r/min for 10 minutes (centrifuge (manufactured by TOMY)) to remove insoluble substances, and the extract obtained was subjected to reversed phase high-performance liquid chromatography (column=μBondapak C18ψ0.39×300 mm (manufactured by Waters Corporation), solvent=methanol:ethanol:water=3:4:4 (volume ratio), flow rate=0.7 mL/min, column oven temperature=40° C., detection wavelength=210 nm) to quantify the terpene compound contained in the extract. In the quantification, the two kinds of the terpene compounds prepared in Preparation Example described above were used as standard substances. The chromatographic pattern obtained by reversed phase high-performance liquid chromatography is shown in FIG. 1A. The axis of ordinates in FIG. 1A is absorbance (at 210 nm), and the axis of abscissas is elution time (minute).

### Comparative Example 1

### Extraction with Distilled Water

The same procedures as in Example 1 were carried out except in the aspect that distilled water (pH 6.78) was used in place of the 1% aqueous Na₂CO₃ solution to obtain an extract, to quantify the terpene compound in the extract. The chromatographic pattern obtained by reversed phase high-performance liquid chromatography is shown in FIG. 1B. The axis of ordinates in FIG. 1B is absorbance (at 210 nm), and the axis of abscissas is elution time (minute).

### Comparative Example 2

### Extraction with Aqueous KCl Solution

The same procedures as in Example 1 were carried out except in the aspect that a 0.15 M aqueous KCl solution (pH 6.54) was used in place of the 1% aqueous Na₂CO₃ solution to obtain an extract, to quantify the terpene compound in the extract. The chromatographic pattern obtained by reversed phase high-performance liquid chromatography is shown in FIG. 1C. The axis of ordinates in FIG. 1C is absorbance (at 210 nm), and the axis of abscissas is elution time (minute).

### Comparative Example 3

### Extraction with Acidic Solvent

The same procedures as in Example 1 were carried out except in the aspect that a 1% aqueous citric acid solution (pH 2.46) was used in place of the 1% aqueous Na₂CO₃ solution to obtain an extract, to quantify the terpene compound in the extract. The chromatographic pattern obtained by reversed phase high-performance liquid chromatography is shown in FIG. 1D. The axis of ordinates in FIG. 1D is absorbance (at 210 nm), and the axis of abscissas is elution time (minute).

The results of quantification of the terpene compounds of Example 1 and Comparative Examples 1 to 3 are shown together in Table 1.

TABLE 1

| | Ex. 1 | Comp. Ex. 1 | Comp. Ex. 2 | Comp. Ex. 3 |
|---|---|---|---|---|
| | (Extraction Solvent) | | | |
| | (1% Na₂CO₃) | (Distilled Water) | (0.15 M KCl) | (1% Citric Acid) |
| hypsiziprenol A₈ | 25 | Below | Below | Below |
| (μg/mL) | | Quantification | Quantification | Quantification |
| | | Limit | Limit | Limit |
| SBS (μg/mL) | 117 | Below | Below | Below |
| | | Quantification | Quantification | Quantification |
| | | Limit | Limit | Limit |
| Total Terpene | 142 | Below | Below | Below |
| Compounds (μg/mL) | | Quantification | Quantification | Quantification |
| | | Limit | Limit | Limit |

* The lower limit of quantification for hypsiziprenol A₈ is 5 μg/mL, and the lower limit of quantification for SBS is 5 μg/mL.

As shown in Table 1, the extract in the case where the 1% aqueous Na₂CO₃ solution of Example 1 was used as the extraction solvent contained a terpene compound in an amount of 142 μg/mL, in contrast to the extracts of the cases where distilled water, the 0.15 M aqueous KCl solution and the 1% aqueous citric acid solution were each used as the extraction solvents (Comparative Examples 1 to 3), in which the contents of the terpene compounds are each below the quantification limit.

### Example 2

### Extraction with Various Alkaline Solvents and Neutralization of Extracts

### Example 2-1

### Extraction with Aqueous Na, CO₃ Solution

The same procedures as in Example 1 were carried out to obtain an extract, to quantify the SBS in the extract. Subsequently, in order to use the extract for a food or beverage, a citric acid powder was added to neutralize the extract. As to the extract after the neutralization, the sugar content was determined with a digital refractometer Palette (manufactured by ATAGO CO., LTD.). In addition, the tone, the presence or absence of foaming, and the presence or absence of precipitation were visually confirmed. Here, regarding the tone, in addition to the visual confirmation, determination with a spectrophotometer UV-160A (manufactured by Shimadzu Corporation) was also made.

### Example 2-2

### Extraction with Aqueous NaOH Solution

The same procedures as in Example 1 were carried out except in the aspect that a 0.1 N aqueous NaOH solution (pH 11.52) was used in place of the 1% aqueous Na₂CO₃ solution to obtain an extract. The extract was subjected to determination of the SBS content, neutralization, and determination of the sugar content in the same manner as in Example 2-1. Also, tone, foaming and precipitation were observed.

### Example 2-3

### Extraction with Anionic Exchange Resin

The same procedures as in Example 1 were carried out except in the aspect that an aqueous 5% ion exchange resin DIAION SA10AOH (manufactured by Nippon Rensui Co.) suspension (pH 10.54) was used in place of the 1% aqueous Na₂CO₃ solution to obtain an extract. The extract was subjected to determination of the SBS content, neutralization, and determination of the sugar content in the same manner as in Example 2-1. Also, tone, foaming and precipitation were observed. The results for Example 2-1 to Example 2-3 are shown together in Table 2.

TABLE 2

| | Ex. 2-1 | Ex. 2-2 | Ex. 2-3 |
|---|---|---|---|
| | (Extraction Solvent) | | |
| | (1% Na₂CO₃) | (0.1 N NaOH) | (Anionic Exchange Resin) |
| Tone (OD₄₂₀) | Reddish | Reddish | Yellowish |
| | Brown (3.30) | Brown (3.56) | Brown (2.43) |
| SBS Concentration | 127.6 | 133.9 | 110.3 |
| (μg/mL) | | | |
| Sugar Content After | 4.9 | 4.4 | 2.9 |
| Neutralization (Brix %) | | | |
| pH (Before Neutralization) | 10.24 | 11.52 | 10.54 |
| pH (After Neutralization) | 6.54 | 6.40 | 6.52 |
| Foaming | Present | Absent | Absent |
| (After Neutralization) | | | |
| Precipitation | Absent | Present | Absent |
| (After Neutralization) | | | |

As shown in Table 2, even in cases where any one of the alkaline extraction solvents were used, extracts richly containing SBS could be obtained. In addition, in the case where an anionic exchange resin was used in the extraction, the sugar content of the extract was lowered, as compared to cases where other alkalis were used, so that SBS having relatively high purity could be obtained. Further, in the case where the anionic exchange resin was used in the extraction, foaming and precipitation did not take place after the neutralization of the extract. This is advantageous in the aspect of facilitating subsequent handling such as concentration of an extract.

### Example 3

### Extraction with Chilled Alkali

The same procedures as in Example 2-3 were carried out for the extract subjected to extraction at low-temperature conditions (5° C. for 24 hours) using an aqueous 5% ion exchange resin DIAION SA10AOH (manufactured by Nippon Rensui Co.) suspension except that the extraction process is carried out at 5° C. for 24 hours, to obtain an extract, to determine the SBS content. The results are shown in Table 3.

TABLE 3

| | Ex. 3 | Ex. 2-3 |
|---|---|---|
| | (Extraction Solvent) | |
| | (5° C. for 24 hour) | (95° C. for 3 hour) |
| SBS Concentration (μg/mL) | 61.6 | 110.3 |

As a result, it was evident that extracts containing a large amount of SBS were obtained even under the extraction conditions of 5° C. for 24 hours, but the SBS content thereof was lowered as compared to the case where the extraction process was carried out at 95° C. for 3 hours.

### Example 4

### Production of Terpene Compound-Containing Beverage

Fruiting bodies of Lyophyllum ulmarium were sufficiently dried in a Constant Temperature Oven (manufactured by Yamato), and the dried product was then powdered with a Cooking Mill (manufactured by NATIONAL PANASONIC). Fifty grams of the Lyophyllum ulmarium powder and 50 g of ion exchange resin DIAION SA10AOH (manufactured by Nippon Rensui Co.), previously washed with hot water, were mixed in one liter of distilled water. The mixture was kept at a set temperature of 95° C. with an autoclave for 3 hours while continue stirring every hour. Next, the mixture was cooled, to obtain 1.1 liters of a chilled product. Thereafter, the ion exchange resin was removed from the chilled product using a 50 mesh sieve (sieve opening: 300 μm), to obtain about 1 liter of supernatant. Two grams of soybean-derived lecithin was added to the supernatant, and the mixture was stirred at room temperature for 30 minutes.

Next, a pH was adjusted from 10.25 to 6.85 with citric acid. In order to remove extraction residue from the pH adjusting solution, 60 grams of a filtration aid Celite #545 was added to 1 liter of the above-mentioned pH adjusting solution, and the mixture was then subjected to suction filtration with a Büchner funnel (Nutsche) pre-coated with 3 grams of the filtration aid Celite #545. 850 milliliters of the filtrate thus obtained, which was the extract derived from Lyophyllum ulmarium, was used as a terpene compound-containing beverage. The beverage had a reduced sensing of bitterness, as compared to those extracts of Examples 1 to 3, and easily drinkable.

### Example 5

### Extraction 2 with Alkali Solvent

The same procedures as in Example 1 were carried out, to give a dry powder of Lyophyllum ulmarium. Next, 10 L of distilled water was added to 500 g of the dry powder of Lyophyllum ulmarium, and hot water washing was carried out at 95° C. for 1 hour. After the hot water washing, the supernatant was removed by a centrifugation procedure of 4,500 r/min for 30 minutes (centrifuge (manufactured by HITACHI, LTD.)), to give a hot water washing of fruiting bodies of Lyophyllum ulmarium. Next, 10 L of distilled water and 500 g of an ion exchange resin DIAION SA10AOH (manufactured by Nippon Rensui Co.), previously washed with hot water, were added to the hot water washing of fruiting bodies of Lyophyllum ulmarium, and the mixture was heat-treated at 95° C. for 3 hours. After the heat treatment, the ion exchange resin was removed from the chilled product with a 50 mesh sieve (sieve opening: 300 μm), to obtain about 10 liters of the supernatant. Twenty grams of soybean-derived lecithin was added to the supernatant, and the mixture was stirred at room temperature for 30 minutes. Next, the pH was adjusted from 9.75 to 6.44 with citric acid. This pH-adjusted solution was subjected to a centrifugation procedure of 4,500 r/min for 20 minutes (centrifuge (manufactured by HITACHI, LTD.) to remove insoluble substance. One-hundred grams of a filtration aid Silika #600S was added to 10 liters of the supernatant of the pH-adjusted solution obtained, and the mixture was then subjected to suction filtration with a Büchner funnel (Nutsche) pre-coated with 100 grams of the filtration aid Silika #600S. 9500 milliliters of the filtrate thus obtained was concentrated with a rotary evaporator, and the concentrate was lyophilized, to obtain an extract derived from Lyophyllum ulmarium.

### Example 6

### Suppressive Activity for Proliferation of Tumor by Extract Derived from Lyophyllum ulmarium

As to CDF1 mice (Japan SLC, Inc.), those female mice of 6-week old were purchased to be used. The tumor cells of IMC carcinoma (hereinafter referred to as IMC) were transplanted to the abdominal cavity of the mice to generate ascites, and the ascites were transplanted to other mice every 7 days, whereby the tumor cells were passaged. The ascites on the seventh day after the passage were collected, and subjected to centrifugal washing with a buffer PHOSPHATE BUFFERED SALTS (TAKARA BIO, INC.), and suspended in the same buffer, and the cell count was taken. Thereafter, the concentration was adjusted to be 5×10⁷ cells/mL. A 0.1 mL portion of this suspension was transplanted subcutaneously to the right side of the abdominal region, and the size of the solid tumor after 7 days was measured. The mice were grouped in 10 mice per group so that the average of the sizes of the tumors will be even in each group.

Next, the extract derived from Lyophyllum ulmarium of Example 5 was mixed with an ordinary powder feed CE-2 so that a weight ratio of the extract was 1.4%, and the mixture was given to the mice. In addition, as a comparative control, an Agaricus-derived extract (SENSEIRO Extracted Gold (SUNDORY Co., Ltd.) was mixed with a powder CE-2 feed so as to have a weight ratio of 2.4%, the mixture was dried, and the dry product was given to the mice. The doses are as shown in Table 4. The control group was given only to the CE-2. The sizes of the tumors were measured on the fourth week after the transplantation of the IMC cells. In addition, as to the sizes of the tumors, their lengths and breadths were determined to calculate their volumes therefrom in accordance with the following calculation formula to be compared. The results of running two experiments are shown in Table 4.

Tumor Volume(mm³)=(Length)×(Breadth)²/2

In addition, the tumor suppressive activity was calculated in accordance with the following calculation formula.

$$Tumor Suppressive Activity ( \% ) = \frac{\begin{matrix}Tumor Volume of Control Group - \\ Tumor Volume of Extract \text{-} Administered Group\end{matrix}}{Tumor Volume of Control Group} \times 100$$

TABLE 4

| Experi- | | Tumor Volume (mm³) | Tumor |
|---|---|---|---|
| mental | Mouse | (Mean ± Standard | Suppressive |
| No. | (Administered Group, Dose) | Error) | Activity (%) |
| 1 | CE-2 (Control) | 1642 ± 193 | |
| | Agaricus-Derived Extract, 3.3 g/kg | 1352 ± 254 | 17.7 |
| | Lyophyllum ulmarium-Derived | 944 ± 112 | 42.5 |
| | Extract, 2.0 g/kg | | |
| 2 | CE-2 (Control) | 1239 ± 228 | |
| | Agaricus-Derived Extract, | 1463 ± 353 | −18.1 |
| | 3.3 g/kg | | |
| | Lyophyllum ulmarium-Derived | 870 ± 169 | 29.8 |
| | Extract, 0.2 g/kg | | |
| | Lyophyllum ulmarium-Derived | 618 ± 115 | 50.1 |
| | Extract, 2.0 g/kg | | |

As a result, the comparative control, which was the group administered with Agaricus-derived extract did not show hardly any suppression in the proliferation of the IMC solid tumors. On the other hand, the group administered with the Lyophyllum ulmarium-derived extract showed suppression in the proliferation of the IMC solid tumors.

### INDUSTRIAL APPLICABILITY

According to the present invention, the extract derived from Lyophyllum ulmarium richly containing a terpene compound, which is suitable for foods and medicaments, can be inexpensively produced. Also, according to the present invention, the terpene compound can be inexpensively produced. Accordingly, the present invention is especially useful in the fields of foods and medicaments.

### BRIEF DESCRIPTION OF THE DRAWINGS

FIG. 1 is a chart showing the results of reversed phase chromatography of an extract derived from Lyophyllum ulmarium, wherein A, B, C and D respectively denote the results of extraction with an alkaline solvent, extraction with distilled water, extraction with an aqueous KCl solution, and extraction with an acidic solvent.

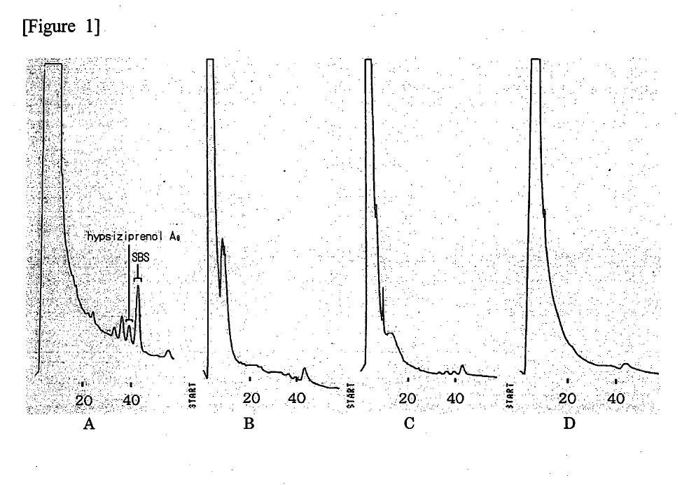

1. A method of producing an extract derived from Lyophyllum ulmarium from fruiting body or mycelium of Lyophyllum ulmarium, wherein the method of producing an extract derived from Lyophyllum ulmarium uses an aqueous alkaline solvent as an extraction solvent.

2. The method according to claim 1, comprising the step of neutralizing the resulting extract with an acid.

3. The method according to claim 1, further comprising the step of adding an emulsifying agent.

4. The method according to claim 1, wherein the aqueous alkaline solvent is an aqueous sodium carbonate solution, an aqueous sodium hydroxide solution, or an anionic exchange resin suspension.

5. The method according to claim 1, wherein the extraction with an extraction solvent is carried out at a temperature of from 40° to 120° C.

6. An extract derived from Lyophyllum ulmarium, obtainable by the method as defined in claim 1.

7. The extract derived from Lyophyllum ulmarium according to claim 6, wherein a terpene compound is contained in an amount of 0.4% by weight or more on a dry solid basis.

8. A food comprising the extract derived from Lyophyllum ulmarium as defined in claim 6 or 7.

9. A medicament comprising the extract derived from Lyophyllum ulmarium as defined in claim 6 or 7.

10. A method of producing a terpene compound, which comprises the following steps (a) and (b):
(a) processing a fruiting body or mycelium of Lyophyllum ulmarium with an aqueous alkaline solvent, to give an extract derived from Lyophyllum ulmarium; and
(b) purifying the terpene compound from the extract obtained in the step (a).

11. The method according to claim 10, wherein the aqueous alkaline solvent is an aqueous sodium carbonate solution, an aqueous sodium hydroxide solution, or an anionic exchange resin suspension.

12. The method according to claim 10 or 11, wherein the processing with an aqueous alkaline solvent is carried out at a temperature of from 40° to 120° C.
